(19) United States
(12) Patent Application Publication
(10) Pub. No.: US 2024/0382246 A1
(43) Pub. Date: Nov. 21, 2024

(54) ISOLATED PRESSURE MONITORING FOR CRYOGENIC BALLOON CATHETER

(71) Applicant: Medtronic Ireland Manufacturing Unlimited Company, Dublin (IE)

(72) Inventors: Carlos H. Lima, Santa Rosa, CA (US); Christopher P. Hartman, Santa Rosa, CA (US); Manoj Kumar Singh, Santa Rosa, CA (US)

(21) Appl. No.: 18/577,336

(22) PCT Filed: Jul. 19, 2022
(86) PCT No.: PCT/EP2022/070218

§ 371 Date: Jan. 8, 2024

Related U.S. Application Data
(60) Provisional application No. 63/226,918, filed on Jul. 29, 2021.

(51) Int. Cl. A61B 18/02; A61B 18/00; A61M 25/00; A61M 25/01; A61M 25/10

(52) U.S. Cl. CPC A61B 18/02; A61M 25/005; A61M 25/0108; A61M 25/10184; A61B 2018/0022; A61B 2018/00511; A61B 2018/00577; A61B 2018/00821; A61B 2018/0212; A61M 2205/3331; A61M 2205/3368

(57) ABSTRACT

A medical device including an elongated shaft having a proximal end, a distal end, and a lumen therethrough. An expandable element is disposed proximate the distal end about the elongated shaft and in fluid communication with the lumen. An inner shaft is disposed within the lumen and within a portion of the expandable element, the inner shaft defining an exhaust port, an infusion port, and a pressure monitoring port. The pressure monitoring port is spaced from both the infusion port and the exhaust port and/or fluidly isolated from the both the infusion port and the exhaust port within the inner shaft. A pressure monitoring tube is disposed within the inner shaft and configured to measure a fluid pressure within the expandable element, with a distal end of the pressure monitoring tube terminating within the expandable element adjacent the pressure monitoring port.

### FIELD

The present technology is generally related to cryogenic balloon catheters, and in particular, to isolating a pressure monitoring tube.

### BACKGROUND

Cryotherapy is a useful treatment modality for many types of medical procedures. In some cases, it is desirable to administer cryotherapy from within a patient's body, such as from within a body lumen. Internal administration of cryotherapy can be advantageous, for example, in at least some neuromodulation procedures. These procedures can include percutaneously introducing a cryotherapeutic element into a patient and then advancing a catheter shaft carrying the cryotherapeutic element along an intravascular path to a suitable treatment location. Once positioned at the treatment location, the cryotherapeutic element can be cooled to modulate nearby nerves. The cooling caused by the cryotherapeutic element, for example, can reduce undesirable local or systemic sympathetic neural activity and thereby achieve various therapeutic benefits.

### SUMMARY

This disclosure generally relates to cryogenic balloon catheters, and in particular, isolating a pressure monitoring tube.

Cryogenic catheters can be configured with an expandable element in fluid communication with refrigerant source to cryogenically ablate a target tissue region. A pressure monitoring tube can be included within the expandable element and configures for measuring a fluid pressure within the expandable element. Some aspects of the present disclosure involve the recognition that in cryogenic balloon catheters in which a pressure monitoring tube terminates at the location of an exhaust port, a negative pressure may be created that can result in incorrect pressure measurements when refrigerant is exhausted through the exhaust port.

In one aspect, a medical device includes an elongated shaft having a proximal end, a distal end, and a lumen therethrough. An expandable element is disposed proximate the distal end about the elongated shaft and in fluid communication with the lumen. An inner shaft is disposed within the lumen and within a portion of the expandable element, the inner shaft defining an exhaust port, an infusion port, and a pressure monitoring port spaced from the exhaust port and the infusion port. A pressure monitoring tube is disposed within the inner shaft and configured to measure a fluid pressure within the expandable element, a distal end of the pressure monitoring tube terminating within the expandable element adjacent the pressure monitoring port.

In another aspect of this embodiment, the inner shaft includes a proximal radiopaque marker band and a distal radiopaque marker band, the proximal radiopaque marker band and the distal radiopaque marker band being disposed about the inner shaft within the expandable element, and wherein the pressure monitoring port is defined within the distal radiopaque marker band.

In another aspect of this embodiment, the inner shaft is a braided for reinforcement.

In another aspect of this embodiment, the distal end of the pressure monitoring tube is angled with respect to the inner shaft and extends outward from the pressure monitoring port.

In another aspect of this embodiment, the expandable element includes a proximal chamber and a distal chamber in fluid communication with each other, and wherein the inner shaft extends into the proximal chamber and the distal chamber.

In another aspect of this embodiment, the inner shaft is fixed with respect to the elongated shaft, and wherein the pressure monitoring port is disposed within the distal chamber, the exhaust port is disposed within the proximal chamber, and the infusion port is disposed distal to the exhaust port and proximal to the pressure monitoring port.

In another aspect of this embodiment, the pressure monitoring tube terminates within the distal chamber.

In another aspect of this embodiment, the device further includes an infusion tube disposed within the elongated shaft, the infusion tube configured to deliver refrigerant to the expandable element and terminating adjacent the infusion port.

In another aspect of this embodiment, the exhaust port includes a plurality of exhaust ports circumferentially disposed about the inner shaft.

In another aspect of this embodiment, the device further includes a thermocouple wire disposed within the inner shaft and extending outward from one of the ports.

In another aspect of this embodiment, the pressuring monitoring port includes a plurality of pressure monitoring ports circumferentially disposed about the inner shaft.

In another aspect of this embodiment, the exhaust port includes a plurality of exhaust ports circumferentially disposed about the inner shaft, and the plurality of exhaust ports and the plurality of pressure monitoring ports are aligned along a longitudinal axis of the inner shaft and offset from the infusion port.

In one aspect, a medical device includes an elongated shaft having a proximal end, a distal end, and a lumen therethrough. An expandable element is disposed proximate the distal end about the elongated shaft and in fluid communication with the lumen. An inner shaft is disposed within the lumen and within a portion of the expandable element, the inner shaft defining an exhaust port, an infusion port, and a pressure monitoring port. A pressure monitoring tube is disposed within the inner shaft and in fluid communication with the pressure monitoring port, the pressure monitoring configured for measuring a fluid pressure within the expandable element, a distal end of the pressure monitoring tube terminating within the expandable element. The infusion port, exhaust port, and pressure monitoring port each being fluidly isolated from each other within the inner shaft.

In another aspect of this embodiment, the device further includes sealing element within the inner shaft, the sealing element being configured to fluidly isolate the infusion port, exhaust port, and the port from each other.

In another aspect of this embodiment, the sealing element is disposed between the pressuring monitoring port and both the exhaust port and the infusion port.

In another aspect of this embodiment, the device further includes a proximal radiopaque marker band and a distal radiopaque marker band disposed along the inner shaft within the expandable element, and wherein the sealing element is disposed between the proximal radiopaque marker band and the distal radiopaque marker band.

In another aspect of this embodiment, the sealing element extends an entire distance between the proximal radiopaque marker band and the distal radiopaque marker band.

In another aspect of this embodiment, the sealing element includes a bonding agent, and wherein the bonding agent is an ultraviolet cured epoxy.

In another aspect of this embodiment, the sealing element is configured to fluid isolate a portion of the inner shaft distal to the sealing element from a portion of the inner shaft proximal to the sealing element.

In another aspect of this embodiment, the distal end of the pressure monitoring tube is angled with respect to the inner shaft and extends outward from the pressure monitoring port.

In another aspect of this embodiment, the expandable element includes a proximal chamber and a distal chamber in fluid communication with each other, and wherein the inner shaft extends into the proximal chamber and the distal chamber.

In another aspect of this embodiment, the inner shaft is fixed with respect to the elongated shaft, and wherein the pressure monitoring port is disposed within the distal chamber and the exhaust port is disposed within the proximal chamber.

In another aspect of this embodiment, the pressure monitoring tube terminates within the distal chamber.

In another aspect of this embodiment, the device includes an infusion tube disposed within the elongated shaft, the infusion tube configured to deliver refrigerant to the expandable element and terminating adjacent the infusion port.

In another aspect of this embodiment, the device includes a thermocouple wire disposed within the inner shaft an extending outward from the infusion port.

Further disclosed herein is a medical device including an elongated shaft having a proximal end, a distal end, and a lumen therethrough, wherein an expandable element is disposed proximate the distal end about the elongated shaft and in fluid communication with the lumen, wherein an inner shaft is disposed within the lumen and within a portion of the expandable element, the inner shaft defining an exhaust port, an infusion port, and a pressure monitoring port, wherein the pressure monitoring port is spaced from both the infusion port and the exhaust port and/or fluidly isolated from the both the infusion port and the exhaust port within the inner shaft, and wherein a pressure monitoring tube is disposed within the inner shaft and configured to measure a fluid pressure within the expandable element, with a distal end of the pressure monitoring tube terminating within the expandable element adjacent the pressure monitoring port.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a system view of an exemplary cryogenic medical system and medical device constructed in accordance with the principles of the present application, with an expandable member shown in cross-section;

FIG. 2A is a side external view of the inner shaft of the medical device shown in FIG. 1, with an expandable member and inner shaft shown in cross section;

FIG. 2B. is a side inside view of the inner shaft of the medical device shown in FIG. 1, with an expandable member, marker bands, and inner shaft shown in cross section;

FIG. 3 is a side inside view of alternative embodiment of the medical device shown in FIG. 2, with an expandable member and inner shaft shown in cross-section;

FIG. 4 is an enlarged side inside view of alternative embodiment of the medical device shown in FIG. 3, with an expandable member and inner shaft shown in cross-section;

FIG. 5 is a side inside view of another embodiment of the distal portion of the medical device shown in FIG. 1, with an expandable member, marker bands, and inner shaft shown in cross section; and

FIG. 6 is a side inside view of another embodiment of the distal portion of the medical device shown in FIG. 1, with an expandable member, marker bands, and inner shaft shown in cross section.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

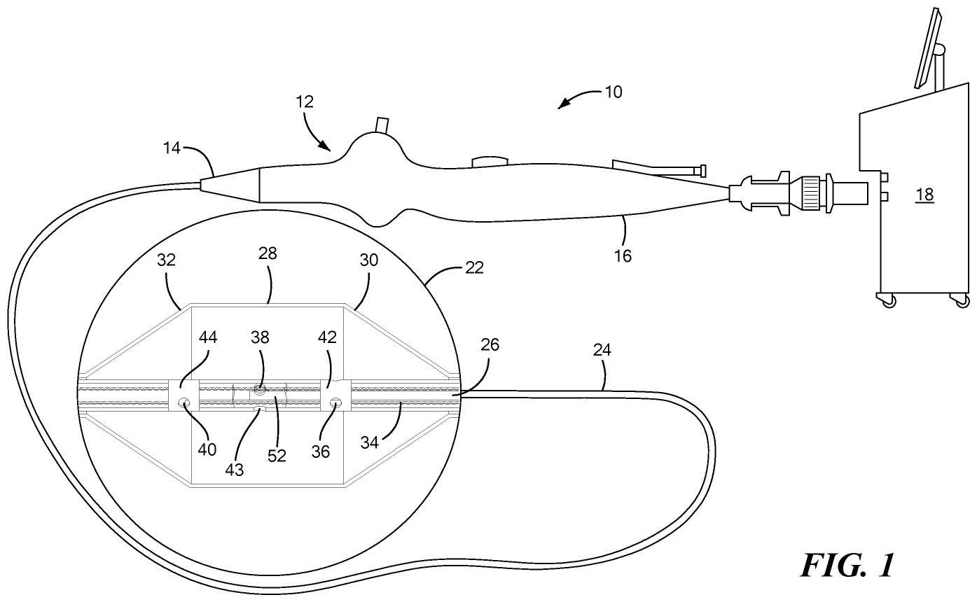

Referring now to the drawings in which like reference designators refer to like elements, there is shown in FIG. 1 an exemplary medical system constructed in accordance with the principles of the present application and designated generally as “10.” The system may include a medical device 12 including a proximal end 14 in fluid communication with a handle 16, which is further in fluid communication with a console 18 having a refrigerant source 20. The console 18 is configured to deliver refrigerant, such as nitrous oxide, to the medical device 12.

Referring now to FIGS. 1-4, the medical device 12 may further include a distal end 22 about which cryogenic energy is exchanged between the medical device 12 and a target tissue, for example, renal tissue (e.g., for renal denervation). For example, the medical device 12 may include an elongated shaft 24 disposed between the proximal end 14 and the distal end 22 and which further defines a lumen 26 there through. An expandable element 28 is disposed proximate the distal end 22 about the elongated shaft 24 and in fluid communication with the lumen 26. The expandable element 28 may be, for example, an inflatable balloon configured to exchange thermal energy with tissue. The expandable element 28 may include a proximal chamber 30 and a distal chamber 32 as discussed in more detail below. An inner shaft 34 is deposed within the lumen 26 and within a portion of the expandable element 28. For example, the inner shaft 34 may extend through both the proximal chamber 30 and the distal chamber 32.

The inner shaft 34, which in some configurations may be braided for reinforcement or multi-layered, defines at least one exhaust port 36, at least one infusion port 38, and at least one pressure monitoring port 40. The pressure monitoring port 40 may be spaced from and/or isolated within the inner shaft from the infusion and exhaust ports to avoid or diminish negative effects from fluid movement through the infusion and exhaust ports on the accuracy of measuring pressure within the balloon. In some configurations, for example as illustrated in FIGS. 1-4, the at least one infusion port 38 may be located distal to the at least one exhaust port 36, and the at least one pressure monitoring port 40 may be located distal to the at least one infusion port 38. However, variations in the relative positions of the ports may vary among embodiments.

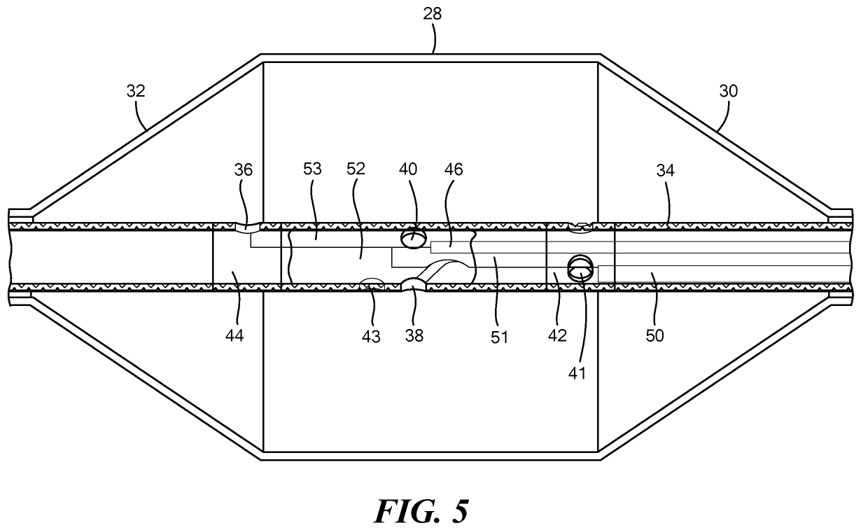

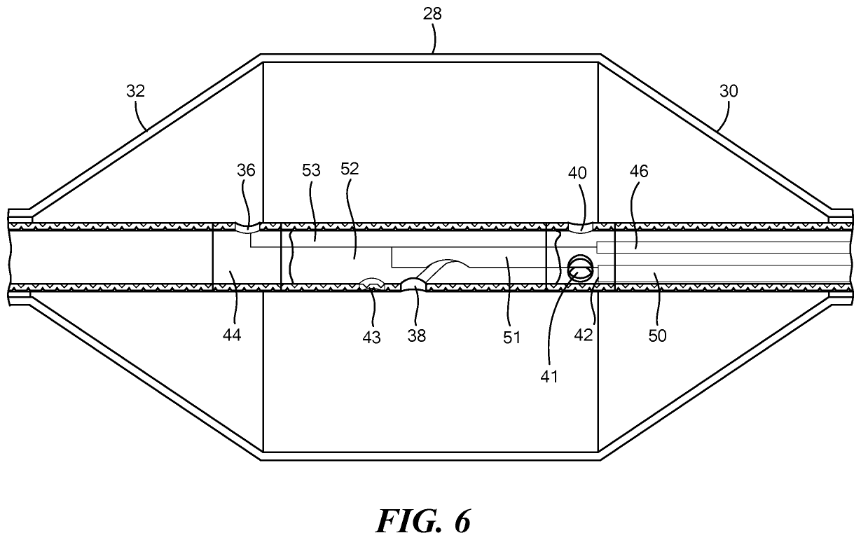

In some configurations, illustrated as an example in FIGS. 1-4, the inner shaft 34 includes a proximal radiopaque marker band 42 and a distal radiopaque marker band 44. The proximal radiopaque marker band 42 and the distal radiopaque marker band 44 are each disposed about the inner shaft 38 within the expandable element 28. The at least one pressure monitoring port 40 may be defined within the distal radiopaque marker band 44 and the at least one exhaust port 36 may be defined within the proximal radiopaque marker band 42. In other configurations, the at least one pressure monitoring port 40 may be distal to or proximal to the distal radiopaque marker band 44 within the distal chamber 32. Similarly, in some configurations, the at least one exhaust port 36 may be distal to or proximal to the proximal radiopaque marker band 42 within the proximal chamber 30. Disposed between the proximal and distal radiopaque marker bands 42, 44 is the at least one infusion port 38. In particular, disposed within the lumen 26 may be an infusion tube 51 which terminates adjacent the infusion port 38 and is configured to transport and deliver refrigerant out through the infusion port 38. The infusion tube 51 may angle outward to direct refrigerant into the expandable element 28. In some configurations, the exhaust port 36 can be in fluid communication with the lumen 26 for removal of refrigerant from the balloon through the space in lumen 26 outside of tubes within the lumen 26 (e.g., infusion tube 51 and pressure monitoring tube 46). In some configurations, the exhaust port 36 can be in fluid communication with an exhaust tube 53 (see, e.g., FIGS. 5-6) for removal of refrigerant from the balloon.

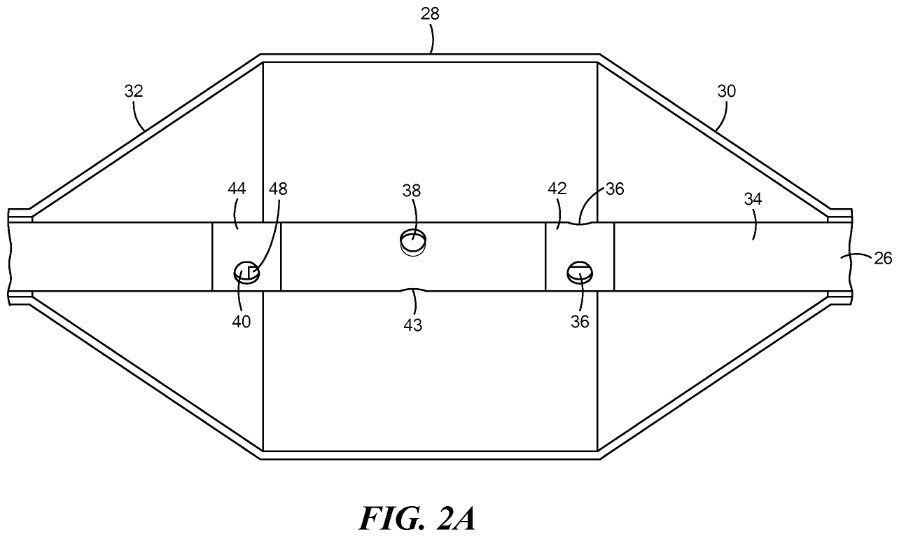

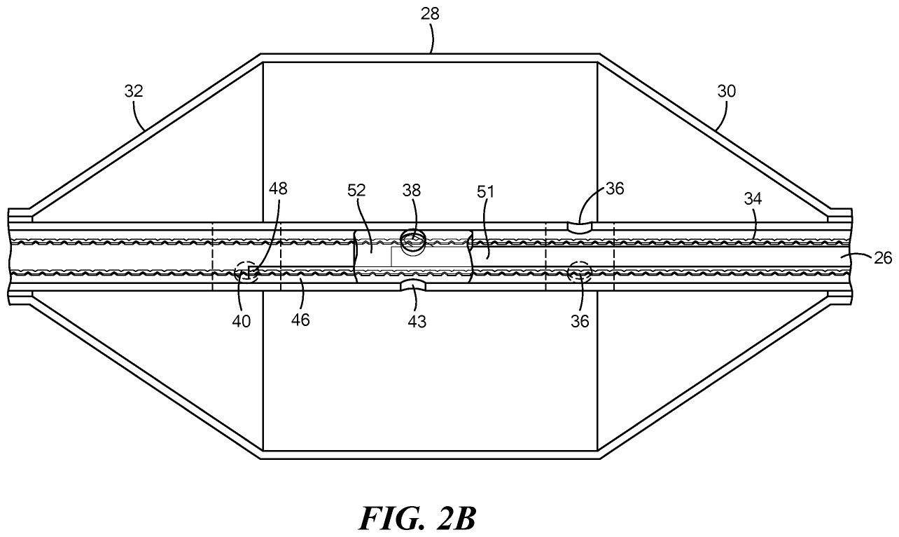

Continuing to refer to FIGS. 2A-4, a pressure monitoring tube 46 is disposed within the inner shaft 34 and is configured to measure a fluid pressure within the expandable element 28. In particular, the pressure monitoring tube 46 is coupled to the handle 16 which is further coupled to the console 18 for measuring a fluid pressure within the expandable element 28. A distal end 48 of the pressure monitoring tube 46 terminating within the expandable element 28 adjacent the at least one pressure monitoring port 40. In some configurations, the pressure monitoring tube 46 is extended through the proximal chamber 30 and into the distal chamber 32, where it terminates in fluid communication with, and optionally also adjacent to, the at least one pressure monitoring port 40. Such a configuration positions the pressure monitoring tube 46 in an unused portion of the expandable element 28, namely the distal chamber 32, and separates the at least one exhaust port 36 from the pressure monitoring tube 46 and the at least one infusion portion 38, proximity to which may interfere with pressure measurements. For example, if the pressure monitoring tube 46 terminated adjacent the at least one exhaust port 36, as refrigerant media is pulled or vacuumed into the at least one exhaust port 36 this may cause an incorrect measurement of pressure within the expandable element 28. Moving the pressure monitoring tube 46 away (e.g., distally) from the at least one exhaust port 36 mitigates this concern.

As shown for example in FIG. 2B, to fluidly isolate the pressure monitoring tube 46 from the infusion port 38 and the exhaust port 36, a sealing element 52 may be included within the inner shaft 34 (e.g., within the lumen 26). For example, a bonding agent may be injected within the inner shaft 34 through one of the ports, for example through bonding port 43, to create one or more sealing elements 52 that fluidly seal a segment of the inner shaft 34 from both to the exhaust port 36 and the infusion port 38. In an example configuration wherein the pressuring monitoring tube 46 extends distally of both the exhaust port 36 and the infusion port 38 (e.g., as illustrated FIGS. 1-4), the sealing element 52 may be positioned proximal to the distal end of the pressure monitoring tube 46 and distal the exhaust port 36 and/or the infusion port 38. In an example configuration wherein the pressuring monitoring tube 46 terminates proximally of both the exhaust port 36 and the infusion port 38 (e.g., as illustrated in FIG. 6), the sealing element 52 may be positioned distal to the distal end of the pressure monitoring tube 46 and proximal to both the exhaust port 36 and the infusion port 38. In an example configuration wherein the pressuring monitoring tube 46 terminates proximally of one of the exhaust port 36 and the infusion port 38 and distally of the other, sealing elements 52 can be positioned both proximal and distal to the distal end of the pressure monitoring tube 46 between it and the exhaust port 36 and the infusion port 38. In such configurations, the pressure monitoring port 40 is disposed within a section of the inner shaft 34 fluidly isolated within the device 12 from the other components of the device 12, and fluid cannot flow within the inner shaft between the pressure monitoring port 40 on one hand and on the other hand the exhaust port and the infusion port without exiting and reentering the inner shaft.

The bonding agent may be, for example, an epoxy or UV cured material. In some configurations, the sealing element 52 is added post positioning of the pressure monitoring tube 46 to its distal location with the distal chamber 32 such that the bonding agent cures around the pressure monitoring tube 46. In other configurations, the sealing element 52 may be a plug configured to seal the inner shaft 34 distal of the infusion port 38 and allow the pressure monitoring tube 46 to extend through the plug.

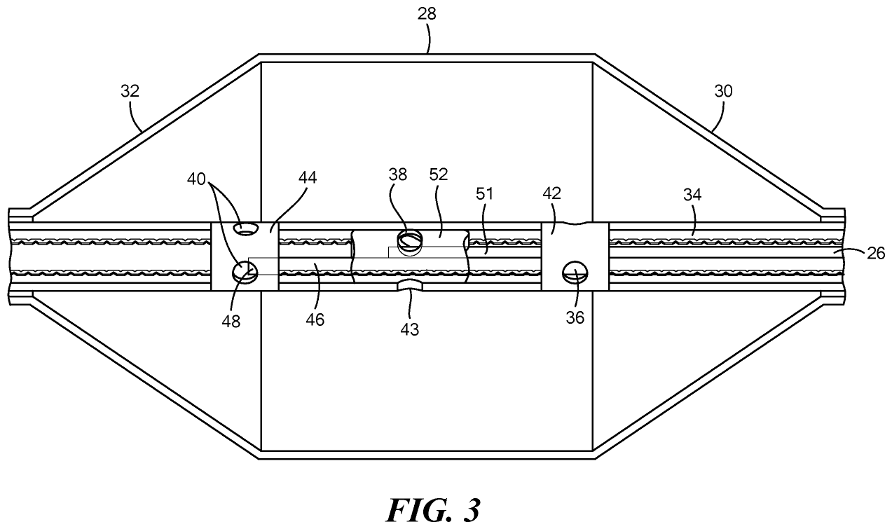

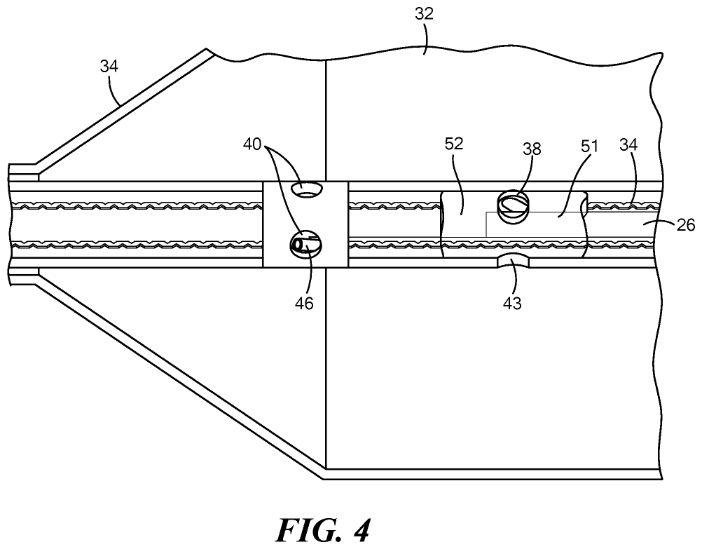

In some configurations, for example as shown in FIG. 2A, a single pressure monitoring port 40 is included offset from the at least one infusion port 38 and radially aligned with the at least one exhaust port 36. In some configurations, for example as shown in FIG. 3, a plurality of pressuring monitoring ports 40 are included and are circumferentially disposed about the inner shaft 34 at the same longitudinal position along the inner shaft 34. In such a configuration, the plurality of pressure monitoring ports 40 aid in the measurement of static pressure within the expandable element 28. Instead of the plurality of pressure monitoring ports 40 being circumferentially disposed about the inner shaft 34 at the same longitudinal position, in another configuration, the plurality of pressure monitoring ports 40 are spaced longitudinally along the length of the shaft at the same or different radial positions about the inner shaft 34. In some configurations, for example as shown in FIG. 4, the pressure monitoring tube 46 may extend into or outward from one of the plurality of pressure monitoring ports 40 at an angle with respect to the major longitudinal axis of the inner shaft 34. In each of the above configurations described or illustrated with reference to FIGS. 1-4, a thermocouple wire 50 (for example as shown in FIGS. 5-6) may be positioned adjacent to or extend outward from a thermocouple wire port 41 or be adjacent to the infusion tube 51. The thermocouple wire 50 may be configured to measure a temperature with the expandable element 28. In other configurations, the thermocouple wire 50 may be positioned adjacent to or extend outward from one of the plurality of pressure monitoring ports 40. The configurations shown or described with reference to FIGS. 3 and 4 can be the same as described elsewhere in this disclosure except for the described differences.

FIGS. 5 and 6 illustrate example configurations in which at least one exhaust port 36 is positioned distally of the infusion port 38 and the pressure monitoring port 40. Except as otherwise noted, the configurations shown or described with reference to FIGS. 5-6 can be the same as described elsewhere in this disclosure. FIG. 5 illustrates an example configuration in which the infusion port 38 and the pressure monitoring port 40 are at the same longitudinal location along the inner shaft 34 while being separated from each other around the circumference of the inner shaft. In some such configurations, the infusion port 38 and the pressure monitoring port 40 may be separated from each other around the circumference of the inner shaft by more than 90 degrees and in some instances by 120 to 180 degrees. Separation of the pressure monitoring tube 40 from the infusion port 38 (e.g., circumferentially) and from the exhaust port 36 (e.g. longitudinally) can avoid or diminish negative effects from fluid movement through the infusion and exhaust ports on the accuracy of measuring pressure within the balloon. The pressure monitoring port 40 also can be circumferentially separated from one or both of the infusion port 38 and the exhaust port 36 in some configurations in which they are also separated longitudinally. Some configurations additionally or alternatively include one or more sealing elements 52 separating the pressure monitoring port 40 from the exhaust port 36 and/or the infusion port 38. In the configuration illustrated in FIG. 5, the sealing element 52 extends distally to a location proximate the exhaust port 36 and distal to both the infusion port 38 and the pressure monitoring port 40 and also extends proximally of both the infusion port 38 and the pressure monitoring port 40, isolating the pressure monitoring tube 40 from the exhaust port 36 and infusion port 38 within the inner shaft 34.

FIG. 6 illustrates an example configuration in which the pressure monitoring port 40 is positioned proximally of and separated from both the infusion port 38 and the exhaust port 36, with the exhaust port 36 being positioned distal to the infusion port 38.

Separation of the pressure monitoring tube 40 from the infusion port 38 and the exhaust port 36 can avoid or diminish negative effects from fluid movement through the infusion and exhaust ports on the accuracy of measuring pressure within the balloon. Some configurations additionally or alternatively include one or more sealing elements 52 separating the pressure monitoring port 40 from the exhaust port 36 and/or the infusion port 38. In the configuration illustrated in FIG. 6, the sealing element 52 extends distally to a location between the exhaust port 36 and the infusion port 38 and proximally to a location between the pressure monitoring port 40 and the infusion port 38, isolating the pressure monitoring tube 40 from the exhaust port 36 and infusion port 38 within the inner shaft 34. As shown in FIG. 6 for example, in some configurations the pressure monitoring port 40 can be positioned at the same longitudinal position as a port 41 associated with the thermocouple wire 50, but at a different circumferential position.

In configurations in which the exhaust port is located distally of the pressure monitoring port 40, such as the configurations illustrated in FIGS. 5 and 6 for example, an exhaust tube 53 may be in fluid communication with the exhaust port 36 or the sealing member 52 may seal around the pressure monitoring tube 46 and pressure monitoring port 40 while allowing refrigerant being exhausted to flow proximally from the exhaust port past the pressure monitoring port 40 to exits the expandable member 28.

As illustrated for example in the configurations shown in FIGS. 5-6, the sealing element 52 may extend the width between the marker bands 42 and 44 (FIG. 6) or may extend from proximate the distal marker band 44 a majority of the length to the proximal marker band 42 (FIG. 5). Moreover, a variety different configurations and placements of the various structures herein may be isolated by extending the sealing element 52.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

Further disclosed herein is the subject-matter of the following clauses:
1. A medical device, comprising:
   an elongated shaft having a proximal end, a distal end, and a lumen therethrough;
   an expandable element disposed proximate the distal end about the elongated shaft and in fluid communication with the lumen;
   an inner shaft disposed within the lumen and within a portion of the expandable element, the inner shaft defining an exhaust port, an infusion port, and a
   pressure monitoring port spaced from the exhaust port and the infusion port; and a pressure monitoring tube disposed within the inner shaft and configured to measure a fluid pressure within the expandable element, a distal end of the pressure monitoring tube terminating within the expandable element adjacent the pressure monitoring port.
2. The device of clause 1, wherein the inner shaft includes a proximal radiopaque marker band and a distal radiopaque marker band, the proximal radiopaque marker band and the distal radiopaque marker band being disposed about the inner shaft within the expandable element, and wherein the pressure monitoring port is defined within the distal radiopaque marker band.
3. The device of clause 1 or of any of the preceding clauses, wherein the inner shaft is a braided for reinforcement.
4. The device of clause 1 or of any of the preceding clauses, wherein the distal end of the pressure monitoring tube is angled with respect to the inner shaft and extends outward from the pressure monitoring port.
5. The device of clause 1 or of any of the preceding clauses, wherein the expandable element includes a proximal chamber and a distal chamber in fluid communication with each other, and wherein the inner shaft extends into the proximal chamber and the distal chamber.
6. The device of clause 5, wherein the inner shaft is fixed with respect to the elongated shaft, and wherein the pressure monitoring port is disposed within the distal chamber, the exhaust port is disposed within the proximal chamber, and the infusion port is disposed distal to the exhaust port and proximal to the pressure monitoring port.
7. The device of clause 6, wherein the pressure monitoring tube terminates within the distal chamber.
8. The device of clause 1 or of any of the preceding clauses, further including an infusion tube disposed within the elongated shaft, the infusion tube configured to deliver refrigerant to the expandable element and terminating adjacent the infusion port.
9. The device of clause 1 or of any of the preceding clauses, wherein the exhaust port includes a plurality of exhaust ports circumferentially disposed about the inner shaft.
10. The device of clause 1 or of any of the preceding clauses, further including a thermocouple wire disposed within the inner shaft and extending outward from one of the ports.
11. The device of clause 1 or of any of the preceding clauses, wherein the pressuring monitoring port includes a plurality of pressure monitoring ports circumferentially disposed about the inner shaft.
12. The device of clause 11, wherein the exhaust port includes a plurality of exhaust ports circumferentially disposed about the inner shaft, and the plurality of exhaust ports and the plurality of pressure monitoring ports are aligned along a longitudinal axis of the inner shaft and offset from the infusion port.
13. A medical device, comprising:
   an elongated shaft having a proximal end, a distal end, and a lumen therethrough;
   an expandable element disposed proximate the distal end about the elongated shaft and in fluid communication with the lumen;
   an inner shaft disposed within the lumen and within a portion of the expandable element, the inner shaft defining an exhaust port, an infusion port, and a pressure monitoring port; and
   a pressure monitoring tube disposed within the inner shaft and in fluid communication with the pressure monitoring port, the pressure monitoring configured for measuring a fluid pressure within the expandable element, a distal end of the pressure monitoring tube terminating within the expandable element; and
   the infusion port, exhaust port, and pressure monitoring port each being fluidly isolated from each other within the inner shaft.
14. The device of clause 13, further including a sealing element within the inner shaft, the sealing element being configured to fluidly isolate the infusion port, exhaust port, and the port from each other.
15. The device of clause 14, wherein the sealing element is disposed between the pressuring monitoring port and both the exhaust port and the infusion port.
16. The device of clause 14 or of any of clauses 14-15, further including a proximal radiopaque marker band and a distal radiopaque marker band disposed along the inner shaft within the expandable element, and wherein the sealing element is disposed between the proximal radiopaque marker band and the distal radiopaque marker band.
17. The device of clause 16, wherein the sealing element extends an entire distance between the proximal radiopaque marker band and the distal radiopaque marker band.
18. The device of clause 14 or of any of clauses 14-17, wherein the sealing element includes a bonding agent, and wherein the bonding agent is an ultraviolet cured epoxy.
19. The device of clause 14 or of any of clauses 14-18, wherein the sealing element is configured to fluid isolate a portion of the inner shaft distal to the sealing element from a portion of the inner shaft proximal to the sealing element.
20. The device of clause 13 or of any of clauses 13-19, wherein the distal end of the pressure monitoring tube is angled with respect to the inner shaft and extends outward from the pressure monitoring port.
21. The device of clause 13 or of any of clauses 13-20, wherein the expandable element includes a proximal chamber and a distal chamber in fluid communication with each other, and wherein the inner shaft extends into the proximal chamber and the distal chamber.
22. The device of clause 21, wherein the inner shaft is fixed with respect to the elongated shaft, and wherein the pressure monitoring port is disposed within the distal chamber and the exhaust port is disposed within the proximal chamber.
23. The device of clause 22, wherein the pressure monitoring tube terminates within the distal chamber.
24. The device of clause 13 or of any of clauses 13-23, further including an infusion tube disposed within the elongated shaft, the infusion tube configured to deliver refrigerant to the expandable element and terminating adjacent the infusion port.
25. The device of clause 13 or of any of clauses 13-24, further including a thermocouple wire disposed within the inner shaft an extending outward from the infusion port.

1. A medical device, comprising:
an elongated shaft having a proximal end, a distal end, and a lumen therethrough;
an expandable element disposed proximate the distal end about the elongated shaft and in fluid communication with the lumen;
an inner shaft disposed within the lumen and within a portion of the expandable element, the inner shaft defining an exhaust port, an infusion port, and a pressure monitoring port spaced from the exhaust port and the infusion port; and
a pressure monitoring tube disposed within the inner shaft and configured to measure a fluid pressure within the expandable element, a distal end of the pressure monitoring tube terminating within the expandable element adjacent the pressure monitoring port.

2. The device of claim 1, wherein the inner shaft includes a proximal radiopaque marker band and a distal radiopaque marker band, the proximal radiopaque marker band and the distal radiopaque marker band being disposed about the inner shaft within the expandable element, and wherein the pressure monitoring port is defined within the distal radiopaque marker band.

3. The device of claim 1, wherein the inner shaft is a braided for reinforcement.

4. The device of claim 1, wherein the distal end of the pressure monitoring tube is angled with respect to the inner shaft and extends outward from the pressure monitoring port.

5. The device of claim 1, wherein the expandable element includes a proximal chamber and a distal chamber in fluid communication with each other, and wherein the inner shaft extends into the proximal chamber and the distal chamber.

6. The device of claim 5, wherein the inner shaft is fixed with respect to the elongated shaft, and wherein the pressure monitoring port is disposed within the distal chamber, the exhaust port is disposed within the proximal chamber, and the infusion port is disposed distal to the exhaust port and proximal to the pressure monitoring port, wherein optionally the pressure monitoring tube terminates within the distal chamber.

7. The device of claim 1, further including an infusion tube disposed within the elongated shaft, the infusion tube configured to deliver refrigerant to the expandable element and terminating adjacent the infusion port.

8. The device of claim 1, wherein the exhaust port includes a plurality of exhaust ports circumferentially disposed about the inner shaft.

9. The device of claim 1, further including a thermocouple wire disposed within the inner shaft and extending outward from one of the ports.

10. The device of claim 1, wherein the pressuring monitoring port includes a plurality of pressure monitoring ports circumferentially disposed about the inner shaft, wherein optionally the exhaust port includes a plurality of exhaust ports circumferentially disposed about the inner shaft, and the plurality of exhaust ports and the plurality of pressure monitoring ports are aligned along a longitudinal axis of the inner shaft and offset from the infusion port.

11. A medical device, comprising:
an elongated shaft having a proximal end, a distal end, and a lumen therethrough;
an expandable element disposed proximate the distal end about the elongated shaft and in fluid communication with the lumen;
an inner shaft disposed within the lumen and within a portion of the expandable element, the inner shaft defining an exhaust port, an infusion port, and a pressure monitoring port; and
a pressure monitoring tube disposed within the inner shaft and in fluid communication with the pressure monitoring port, the pressure monitoring configured for measuring a fluid pressure within the expandable element, a distal end of the pressure monitoring tube terminating within the expandable element; and
the infusion port, exhaust port, and pressure monitoring port each being fluidly isolated from each other within the inner shaft.

12. The device of claim 11, further including a sealing element within the inner shaft, the sealing element being configured to fluidly isolate the infusion port, exhaust port, and the port from each other.

13. The device of claim 12, wherein the sealing element is disposed between the pressuring monitoring port and both the exhaust port and the infusion port.

14. The device of claim 12, further including a proximal radiopaque marker band and a distal radiopaque marker band disposed along the inner shaft within the expandable element, and wherein the sealing element is disposed between the proximal radiopaque marker band and the distal radiopaque marker band, wherein optionally the sealing element extends an entire distance between the proximal radiopaque marker band and the distal radiopaque marker band.

15. The device of claim 12, wherein the sealing element includes a bonding agent, and wherein the bonding agent is an ultraviolet cured epoxy, and/or wherein the sealing element is configured to fluid isolate a portion of the inner shaft distal to the sealing element from a portion of the inner shaft proximal to the sealing element.

16. The device of claim 11, wherein the distal end of the pressure monitoring tube is angled with respect to the inner shaft and extends outward from the pressure monitoring port.

17. The device of claim 11, wherein the expandable element includes a proximal chamber and a distal chamber in fluid communication with each other, and wherein the inner shaft extends into the proximal chamber and the distal chamber.

18. The device of claim 17, wherein the inner shaft is fixed with respect to the elongated shaft, and wherein the pressure monitoring port is disposed within the distal chamber and the exhaust port is disposed within the proximal chamber, wherein optionally the pressure monitoring tube terminates within the distal chamber.

19. The device of claim 11, further including an infusion tube disposed within the elongated shaft, the infusion tube configured to deliver refrigerant to the expandable element and terminating adjacent the infusion port, and/or further including a thermocouple wire disposed within the inner shaft an extending outward from the infusion port.
